(19) United States
(12) Patent Application Publication
(10) Pub. No.: US 2023/0126775 A1
(43) Pub. Date: Apr. 27, 2023

(54) SYSTEM AND METHODS FOR PERSONALIZED SKIN HEALTH AND/OR APPEARANCE REGIMEN SUPPORTED WITH MIRROR-BASED DIGITAL INTERFACE APPARATUS

(71) Applicant: AQUAVIT PHARMACEUTICALS, INC., New York, NY (US)

(72) Inventor: Sobin Chang, New York, NY (US)

(21) Appl. No.: 17/912,369

(22) PCT Filed: Mar. 16, 2021
(86) PCT No.: PCT/US2021/022617

§ 371 Date: Sep. 16, 2022

Related U.S. Application Data
(60) Provisional application No. 62/990,342, filed on Mar. 16, 2020.

(51) Int. Cl. G16H 40/67; A61B 5/00; G06Q 30/0601

(52) U.S. Cl. CPC G16H 40/67; A61B 5/441; A61B 5/0077; A61B 5/0022; A61B 5/7465; A61B 5/749; A61B 5/748; G06Q 30/0631

(57) ABSTRACT

The present invention provides a system for personalized skin health and/or appearance regimen supported with a device that comprises a mirror-based digital interface apparatus, directly from the device to one's account. In some embodiments, the device utilizes built-in virtual trainers/beauticians to give realtime personalized suggestions, product recommendations, based on the user's preference. The trainer/beautician can provide information through text on the screen or use audio. In some embodiments, it can display personalities, influencers, skin health experts who suggest skin workout regiment and them using the mirror-based digital interface. In some embodiments, realtime questions can be asked and answered with a data library integrated within the device and online database.

### CROSS-REFERENCE TO RELATED APPLICATIONS

This application claims the benefit of the U.S. Provisional Appl. No. 62/990,342, filed on Mar. 16, 2020, the contents of which are hereby incorporated by reference in their entirety.

### SUMMARY

It is to be understood that both the foregoing general description of the embodiments and the following detailed description are exemplary, and this do not restrict the scope of the embodiments.

In one aspect the invention provides system and methods for personalized skin health and/or appearance regimen supported with a device that comprises a mirror-based digital interface apparatus.

It is to be understood that both the foregoing general description and the following detailed description are exemplary, and thus do not restrict the scope of the invention. Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The skilled artisan will understand that the drawings, described below, are for illustration purposes only. The drawings are not intended to limit the scope of the present teachings in any way.

FIG. 1 illustrates the process of how the mirror-based digital interface apparatus functions. The Aquamirror or the mirror-based digital interface analyses the user's skin health and/or appearance through external devices such as cameras, skin analyzer, and other devices as listed in the summary and claims. These devices can also be in-built in the mirror. After collecting information of the skin by the mirror-based digital interface, the data is analyzed by an AI platform that is integrated with a cloud storage program. The data collected is analyzed and stored in the cloud platforms such as iCloud, Google Drive, and other similar cloud storage drives. This information can be accessed by the user through an application that runs on mobile phones, personal computers, etc.

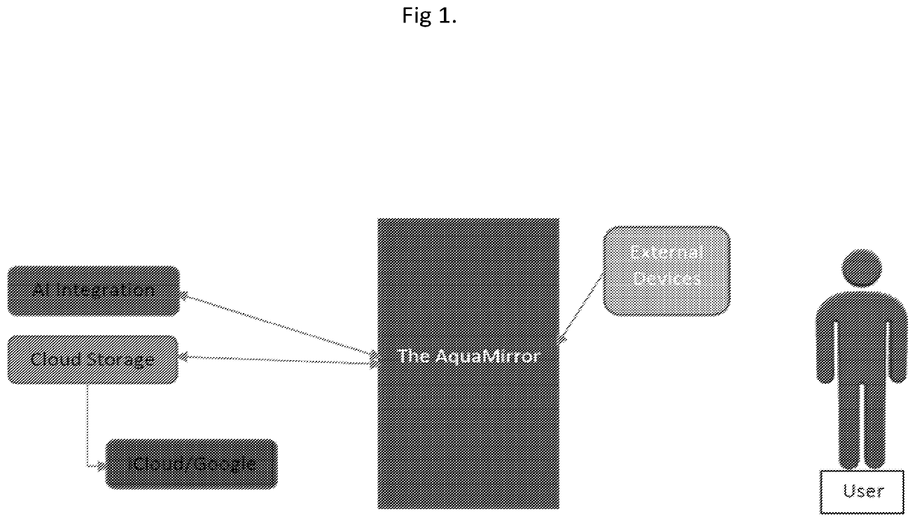

### DETAILED DESCRIPTION

Reference will now be made in detail to embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Furthermore, where the description of one or more embodiments uses the term “comprising,” those skilled in the art would understand that, in some specific instances, the embodiment or embodiments can be alternatively described using the language “consisting essentially of” and/or “consisting of.”

It is contemplated that any system, method or composition described herein can be implemented with respect to any other system, method or composition described herein.

In one embodiment, the invention provides system and method for personalized skin health and/or appearance regimen supported with a device that comprises a mirror-based digital interface apparatus.

In one embodiment, the invention provides a mirror-based digital interface apparatus for use with the systems and methods herein.

In one aspect, the device acts as a mirror for general use to check the user's appearance.

In some embodiments, the screen is a touch-screen, allowing the user to choose various options available on the device.

In some embodiments, the device uses a 8K resolution camera that records the user's whole body skin features. This includes, but is not limited to, the face, neck, shoulder, arm, upper body, lower body, etc.

In some embodiments, the device analyzes the user's skin and provides suggestions to improve the user's skin health, store recorded information to keep track of progress. Skin analyzer is able to detect fine lines, acne, scars, melasma, hyperpigmentation, pores, etc. With the user's preference, the analyzer suggests recommendations to the skincare regimen to achieve the goal set by the user.

In some embodiments, the device integrates with an artificial intelligence system to provide skin health data, expert opinion, related suggestions, etc. The device will be able to store information on the device itself and in the cloud. The storage size is, but not limited to, 8 GBs, 16 GBs, 32 GBs, 64 GBs, etc.

In some embodiments, the device suggests and simulates images of the user's skin to show how the treatment/recommendation/suggestion can impact the skin.

In some embodiments, the device connects to Wi-Fi and stores skin health information in the cloud for reference. This information is protected by a closed circuit system, only accessible by the user's appointed password.

In some embodiments, the device uses HD display to display information about the user's skin, videos, personalized skin care shopping list, etc.

In some embodiments, the device uses a built-in microphone to receive the user's voice to navigate through the device. The device can be activated by voice recognition.

In some embodiments, the device uses built-in speakers to transmit audio. The audio can be, but not limited to, trainer's voice, suggested videos, user's chosen song, etc.

In some embodiments, the device integrates with user's social media accounts. This includes, but is not limited to, facebook, twitter, instagram, tiktok, YouTube, etc. Users can upload photos directly from the device to one's account.

In some embodiments, the device utilizes built-in virtual trainers/beauticians to give real-time personalized suggestions, product recommendations, based on the user's preference. The trainer/beautician can provide information through text on the screen or use audio. In some embodiments, it can display personalities, influencers, skin health experts who suggest skin workout regiment and them using the mirror-based digital interface. In some embodiments, real-time questions can be asked and answered with a data library integrated within the device and online database. The user can look up tips, recommendations, questions that did not come out as primary recommendations.

In some embodiments, the device utilizes a database to give suggestions and recommendations for makeups. Tutorials, recorded sessions, product information from videos help the user learn what type of makeup would fit oneself. Real-time simulations of makeup on oneself using the built-in camera will aid the user on styling of the makeup and personalizing makeup.

In some embodiments, the device connects the user to online skincare websites for personalized purchases on the spot.

In some embodiments, the device connects to the user's personal electronic equipment through Bluetooth, etc. and syncs with the device to navigate through on the user's personal devices. An application will allow the user to navigate through remotely.

In some embodiments, the device uses LED lights to aid user experience of the mirror. The lights are adjustable to fit the mood, color, tone of the user as preferred.

In some embodiments, the device will be able to be placed on the countertop for face-specific use. In other embodiments, the device will be able to be mounted on the wall, leaned against the wall for full body use.

In some embodiments, the device can be activated by the user through a mobile application on smartphone, smartwatch.

In some embodiments, the user can activate the device by a gesture (voice, hand, etc.), touch screen or a power button.

In some embodiments, after activation, a camera with flash is switched on. In some embodiments, the camera records and live streams the user's body on the screen. In some embodiments, the camera analyses facial features and captures data points.

In some embodiments, individuals such as a skin workout beautician, a skin trainer, a model, a social media influencer, or a combination thereof, appears in the mirror-based digital interface.

In some embodiments, these individuals suggest skin workout regimen, product recommendations and home-made product recipe.

In some embodiments, the user can purchase ingredients for a product recipe. In some embodiments, images of ingredients would come on screen and users can opt to purchase it online. In some embodiments, these purchases can be made through e-commerce sites such as Amazon, WholeFoods, etc.

In some embodiments, the mirror-based digital interface can display videos of individuals using the mirror-based digital interface system, or other skincare products.

In some embodiments, the user can choose to not view the skin workout beautician or skin trainer on screen and just opt to use the camera. In some embodiments, the user can opt out of videos and cameras, and just use the mirror as a reflective mirror.

In some embodiments, the user can take photographs and videos that automatically transfer to the user's cloud storage accounts.

In some embodiments, the user can choose to share the photographs and videos on social media platforms. In some embodiments, the user can share the photographs and videos through a voice gesture, hand gesture, touch screen or button, or a combination thereof.

In some embodiments, the user's data is constantly uploaded to the user specified cloud storage.

In some embodiments, the data collected from the user and data repository available in the cloud can aid the virtual beautician/skin trainer to give personalized suggestions to the user. In some embodiments, the data is also shared with physicians such as dermatologists to analyze and prescribe treatment regimen. These suggestions and regimen include but not limited to: Personalized skin care routine, skin care products.

In some embodiments, the user can set up tele-health appointments with dermatologists & physicians. In some embodiments, the virtual appointments with aestheticians and/or healthcare professionals can allow personalized treatment and medication to be prescribed.

In some embodiments, the digital interface displays AI-based suggestions and simulated images to the user's skin to show how each treatment/recommendation can impact their skin. In some embodiments, it will be a simulation of how the products or workout regimen would change their skin. For instance, if the user has stretch marks, the mirror-based digital interface system would recommend a product for stretch marks. And the digital interface would display a simulated image of how the skin would look after using the product.

In some embodiments, the user can shut down the device by a gesture (voice, hand, etc.), touch screen or a power button.

In some embodiments, the invention provides a system for personalized skin health and/or appearance regimen supported with a device that comprises a mirror-based digital interface apparatus.

In some embodiments, the device is a touch-screen interface and acts as a mirror for general use to check the user's appearance.

In some embodiments, the device analyzes the user's skin health and recommends skincare regimen using expert opinion and artificial intelligence to achieve the user's skin goal; wherein the device can optionally analyze dark spots, acnes, scars, hyperpigmentation, fine lines, wrinkles, or other concerns of the user and run through an artificial intelligence algorithm to suggest a skin care regimen personalized for the user using professional opinions and online databases; wherein the user can optionally utilize skincare regimen sessions recommended or create one's own personalized skincare routine; wherein the skin can optionally be analyzed by a built-in high resolution camera and light or user's choice of analyzing device that can be synced with the mirror-based digital interface, wherein the high resolution camera is capable of recording the user's whole body skin features including, but not limited to, the face, neck, shoulder, arm, upper body, lower body, etc.

In some embodiments, the analyzed data is stored in the device or transferred to the cloud, as the user prefers to keep track of the progress and give advanced recommendations, wherein optionally the data will only be available through user's identification to allow secure storage of personal data.

In some embodiments, the device connects through Wi-fi or bluetooth to access user's social media accounts, online shopping sites, etc., to access videos, sessions, photos, products the user is interested in and also upload photos, videos, personal skincare routine to share with others.

In some embodiments, the device utilizes a database to give suggestions and recommendations for makeups, including tutorials, recorded sessions, product information videos.

In some embodiments, the device is activated by touch or voice to meet the user's preference.

In some embodiments, the device is activated by application on the user's personal electronic device to navigate through.

In some embodiments, the device size can vary from fitting on a countertop to a full-body sized device to meet each individual's preference.

In some embodiments, the device is countertop sized to focus on the facial skincare routine.

In some embodiments, the device recommends a skincare regimen to aid the user to achieve one's skincare goals.

In some embodiments, the device is full-body sized and is able to analyze the whole body's skin condition.

In some embodiments, the device recommends a skincare and exercise regimen to aid the user to achieve one's body goals.

In some embodiments, the device analyzes the user's skin and provides suggestions to improve the user's skin health, wherein the device stores recorded information to keep track of progress.

In some embodiments, the device is able to detect fine lines, acne, scars, melasma, hyperpigmentation, pores, and suggests recommendations for personalized skincare regimen to the user.

In some embodiments, the device suggests and simulates images of the user's skin to show how the treatment/recommendation/suggestion can impact the skin.

In some embodiments, the device connects to Wi-Fi and stores skin health information in a password-protected cloud storage.

In some embodiments, the device uses an HD display screen to display information about the user's skin, videos, personalized skin care shopping list, etc.

In some embodiments, the device has a built-in microphone to receive the user's voice to be activated and to navigate through the different screens of the device.

In some embodiments, the device uses built-in speakers to transmit audio, wherein the audio can be, but is not limited to, a trainer's voice, suggested videos, user's chosen song, etc.

In some embodiments, the device utilizes built-in virtual trainers/beauticians to give real-time personalized suggestions, product recommendations, based on the user's preference.

In some embodiments, the device utilizes a database to give suggestions and recommendations for makeups through tutorials, recorded sessions, product information videos.

In some embodiments, the device connects the user to online skin care websites for personalized online purchases.

In some embodiments, the device has various colors of LED lights to match the user's atmosphere, skin tone, or preferences.

In some embodiments, the mirror-based digital interface device is used in telehealth consultations with physicians and facilitate physicians to prescribe medications to the user.

### Sample Embodiments

1. A method for personalized skin health and/or appearance regimen supported with mirror-based digital interface apparatus.
2. The method of claim 1, wherein the device is a touch-screen interface and acts as a mirror for general use to check the user's appearance.
3. The method of paragraph 1, wherein the device analyzes the user's skin health and recommends skincare regimen using expert opinion and AI to achieve the user's skin goal.
   a. The device will analyze dark spots, acnes, scars, hyperpigmentation, fine lines, wrinkles, or other concerns of the user and run through an AI algorithm to suggest a skin care regimen personalized for the user using professional opinions and online databases.
   b. The user can utilize skincare regimen sessions recommended or create one's own personalized skincare routine.
   c. The skin can be analyzed by the built-in 8K resolution camera and light or user's choice of analyzing device that can be synced with the mirror-based digital interface. The 8K resolution camera records the user's whole body skin features including, but not limited to, the face, neck, shoulder, arm, upper body, lower body, etc.
4. The method of paragraph 1, wherein the analyzed data is stored in the device or transferred to the cloud, as the user prefers to keep track of the progress and give advanced recommendations.
   a. The data will only be available through user's identification to allow secure storage of personal data.
5. The method of paragraph 1, wherein the device connects through bluetooth, to access user's social media accounts, online shopping sites, etc., to access videos, sessions, photos, products the user is interested in and also upload photos, videos, personal skincare routine to share with others.
6. The method of paragraph 1, wherein the device utilizes a database to give suggestions and recommendations for makeups, including tutorials, recorded sessions, product information videos.
7. The method of paragraph 1, wherein the device is activated by touch or voice to meet the user's preference. It is activated by application on the user's personal electronic device to navigate through.
8. The device size can vary from fitting on a countertop to a full-body sized device to meet each individual's preference.
   a. Countertop sized devices will focus on the facial skincare routine.
      i. Recommend skincare regimen to aid the user to achieve one's skincare goals.
   b. Full-body sized devices will be able to analyze the whole body's skin condition.
      i. Recommend skincare and exercise regimen to aid the user to achieve one's body goals.
9. The method of paragraph 1, wherein the device analyzes the user's skin and provides suggestions to improve the user's skin health, stores recorded information to keep track of progress.
10. The method of paragraph 1, wherein the device is able to detect fine lines, acne, scars, melasma, hyperpigmentation, pores, and suggests recommendations to personalized skincare regimen to the user.
11. The method of paragraph 1, wherein the device suggests and simulates images of the user's skin to show how the treatment/recommendation/suggestion can impact the skin.
12. The method of paragraph 1, wherein the device connects to Wi-Fi and stores skin health information in a password-protected cloud storage.
13. The method of paragraph 1, wherein the device uses an HD display screen to display information about the user's skin, videos, personalized skin care shopping list, etc.
14. The method of paragraph 1, wherein the device has a built-in microphone to receive the user's voice to be activated and to navigate through the different screens of the device.
15. In some embodiments, the device uses built-in speakers to transmit audio. The audio can be, but not limited to, trainer's voice, suggested videos, user's chosen song, etc.
16. The method of paragraph 1, wherein the device utilizes built-in virtual trainers/beauticians to give real-time personalized suggestions, product recommendations, based on the user's preference.
17. The method of paragraph 1, wherein the device utilizes a database to give suggestions and recommendations for makeups through tutorials, recorded sessions, product information videos.
18. The method of paragraph 1, wherein the device connects the user to online skincare web sites for personalized online purchases.
19. The method of paragraph 1, wherein the device has various colors of LED lights to match the user's atmosphere, skin tone, preference.
20. The method of paragraph 1, wherein the mirror-based digital interface device helps in telehealth consultations with physicians and facilitate physicians to prescribe medications to the users.

I claim:

1. A system for personalized skin health and/or appearance regimen supported with a device that comprises a mirror-based digital interface apparatus.

2. The system of claim 1, wherein the device is a touch-screen interface and acts as a mirror for general use to check the user's appearance.

3. The system of claim 1 or 2, wherein the device analyzes the user's skin health and recommends skincare regimen using expert opinion and artificial intelligence to achieve the user's skin goal; wherein the device can optionally analyze dark spots, acnes, scars, hyperpigmentation, fine lines, wrinkles, or other concerns of the user and run through an artificial intelligence algorithm to suggest a skin care regimen personalized for the user using professional opinions and online databases; wherein the user can optionally utilize skincare regimen sessions recommended or create one's own personalized skincare routine; wherein the skin can optionally be analyzed by a built-in high resolution camera and light or user's choice of analyzing device that can be synced with the mirror-based digital interface, wherein the high resolution camera is capable of recording the user's whole body skin features including, but not limited to, the face, neck, shoulder, arm, upper body, lower body, etc.

4. The system of any of claims 1-3, wherein the analyzed data is stored in the device or transferred to the cloud, as the user prefers to keep track of the progress and give advanced recommendations, wherein optionally the data will only be available through user's identification to allow secure storage of personal data.

5. The system of any of claims 1-4, wherein the device connects through Wi-fi or bluetooth to access user's social media accounts, online shopping sites, etc., to access videos, sessions, photos, products the user is interested in and also upload photos, videos, personal skincare routine to share with others.

6. The system of any of claims 1-5, wherein the device utilizes a database to give suggestions and recommendations for makeups, including tutorials, recorded sessions, product information videos.

7. The system of any of claims 1-6, wherein the device is activated by touch or voice to meet the user's preference.

8. The system of any of claims 1-7, wherein the device is activated by application on the user's personal electronic device to navigate through.

9. The system of any of claims 1-8, wherein the device size can vary from fitting on a countertop to a full-body sized device to meet each individual's preference.

10. The system of any of claims 1-9, wherein the device is countertop sized to focus on the facial skincare routine.

11. The system of any of claims 1-10, wherein the device recommends a skincare regimen to aid the user to achieve one's skincare goals.

12. The system of any of claims 1-11, wherein the device is full-body sized and is able to analyze the whole body's skin condition.

13. The system of any of claims 1-12, wherein the device recommends a skincare and exercise regimen to aid the user to achieve one's body goals.

14. The system of any of claims 1-13, wherein the device analyzes the user's skin and provides suggestions to improve the user's skin health, wherein the device stores recorded information to keep track of progress.

15. The system of any of claims 1-14, wherein the device is able to detect fine lines, acne, scars, melasma, hyperpigmentation, pores, and suggests recommendations for personalized skincare regimen to the user.

16. The system of any of claims 1-15, wherein the device suggests and simulates images of the user's skin to show how the treatment/recommendation/suggestion can impact the skin.

17. The system of any of claims 1-16, wherein the device connects to Wi-Fi and stores skin health information in a password-protected cloud storage.

18. The system of any of claims 1-17, wherein the device uses an HD display screen to display information about the user's skin, videos, personalized skin care shopping list, etc.

19. The system of any of claims 1-18, wherein the device has a built-in microphone to receive the user's voice to be activated and to navigate through the different screens of the device.

20. The system of any of claims 1-19, wherein the device uses built-in speakers to transmit audio, wherein the audio can be, but is not limited to, a trainer's voice, suggested videos, user's chosen song, etc.

21. The system of any of claims 1-20, wherein the device utilizes built-in virtual trainers/beauticians to give real-time personalized suggestions, product recommendations, based on the user's preference.

22. The system of any of claims 1-21, wherein the device utilizes a database to give suggestions and recommendations for makeups through tutorials, recorded sessions, product information videos.

23. The system of any of claims 1-22, wherein the device connects the user to online skin care websites for personalized online purchases.

24. The system of any of claims 1-23, wherein the device has various colors of LED lights to match the user's atmosphere, skin tone, or preferences.

25. The system of any of claims 1-24, wherein the mirror-based digital interface device is used in telehealth consultations with physicians and facilitate physicians to prescribe medications to the user.
